(19) United States
(12) Patent Application Publication
(10) Pub. No.: US 2024/0342452 A1
(43) Pub. Date: Oct. 17, 2024

(54) A MICRONEEDLE APPARATUS

(71) Applicant: Nusmetics Pte. Ltd., Singapore (SG)

(72) Inventors: Duc-Viet NGUYEN, Singapore (SG); Himanshu KATHURIA, Singapore (SG)

(73) Assignee: Nusmetics Pte. Ltd., Singapore (SG)

(21) Appl. No.: 18/293,059

(22) PCT Filed: Jul. 29, 2022
(86) PCT No.: PCT/SG2022/050541

§ 371 Date: Jan. 29, 2024

(30) Foreign Application Priority Data
Jul. 30, 2021 (SG) 10202108366T

(51) Int. Cl. A61M 37/00

(52) U.S. Cl. CPC A61M 37/0015; A61M 2037/0023; A61M 2037/0061

(57) ABSTRACT

There is provided a microneedle apparatus 100 for engaging a surface, the apparatus 100 comprising a housing 104 comprising a socket 106; and a microneedle device 102 moveable within the socket 106, the microneedle device 102 comprising a substantially spherical substrate 102a and a plurality of microneedles 102b projecting from the substrate 102a, the microneedle device 102 being configured to move within the socket 106 and against the surface with 3-dimensional movements such that, in use, the plurality of microneedles 102b engage the surface. There is also provided a method of engaging a surface, such as by using the microneedle apparatus 100.

### TECHNICAL FIELD

The present disclosure relates to a microneedle apparatus for engaging a surface.

### BACKGROUND

Transdermal and/or local delivery of substances such as therapeutic and cosmetic ingredients to skin surfaces provides several advantages over other delivery routes: (i) bypassing first-pass metabolism, gastrointestinal irritation and degradation, (ii) non-invasiveness and (iii) self-administration. However, this route is limited by absorption barriers such as stratum corneum and tight junctions which hinders the penetration of foreign substances and therapeutics. The therapeutics (<500 Da) with suitable lipophilicity are minimally absorbed into skin. The delivery of large molecular weight therapeutics and cosmetic ingredients, such as macromolecules, peptides, insulin, vaccine, etc. is challenging, and most skin products also face this limitation. The skin barrier can be overcome by use of microneedles that creates micro size pathways to enhance the absorption and/or localisation and/or distribution of actives.

Conventional microneedle devices include but not limited to microneedle patch, microneedle stamp, derma roller and derma pens, which are known to enhance skin permeation. However, these devices have limitations such as mostly applicable only for flat surfaces. The application of those on certain skin areas may have incomplete insertion; hence, inefficient and non-uniform delivery. In contrast, the derma roller may be applicable for curved surfaces but its movement is limited by only single-dimensional rolling. Moreover, the derma roller is difficult to reach certain skin surfaces such as eyelids. Furthermore, it is also responsible for infections due to reuse. The derma pens can be applied on small skin areas, and effectively punctures skin using vertical vibration of needles on skin along the needle axis. However, the derma pen needs to be held vertically on the skin surface and moved along the skin contour slowly during application to prevent horizontal cuts/injuries on the skin. As a result, the derma pen has limited movement on skin surface where the movement is not as smooth and flexible as derma rollers.

Therefore, there is a need for a better microneedle apparatus.

### SUMMARY

The present disclosure seeks to address these problems, and/or to provide an improved microneedle apparatus.

According to a first aspect, there is provided a microneedle apparatus for engaging a surface, the apparatus comprising:
a housing comprising a socket; and
a microneedle device moveable within the socket, the microneedle device comprising a substantially spherical substrate and a plurality of microneedles projecting from the substrate,
   wherein the microneedle device is configured to move within the socket and against the surface with 3-dimensional movements such that, in use, the plurality of microneedles engages the surface.

According to a second aspect, there is provided a kit comprising the apparatus according to the first aspect, and a substance, a mixture, or a formulation.

According to a third aspect, there is provided a method of engaging a surface, the method comprising:
handling a microneedle apparatus comprising a housing with a socket;
moving a microneedle device within the socket and against the surface with 3-dimensional movements, the microneedle device comprising a substantially spherical substrate and a plurality of microneedles projecting from the substrate; and
engaging the surface via the plurality of microneedles.

### BRIEF DESCRIPTION OF THE DRAWINGS

In order that the present disclosure may be fully understood and readily put into practical effect there shall now be described by way of non-limitative example only exemplary embodiments, the description being with reference to the accompanying illustrative drawings.

FIG. 1 shows a schematic of an exemplary embodiment of the microneedle apparatus.

FIG. 2 shows a schematic of a magnetic holding mechanism to hold the microneedle device within the socket and allow the microneedle device to move in 3D directions.

FIG. 3 shows a schematic of a chemical and physical holding mechanism using an adhesive or supportive layer to hold the microneedle device within the socket and allow the microneedle device to move in 3D directions.

FIG. 4 shows a schematic of exemplary embodiments of the microneedle apparatus comprising a plurality of sockets, wherein a microneedle device having a substantially spherical substrate comprising a plurality of microneedles projecting from the substrate is held in each of the plurality of sockets.

FIG. 5 shows a schematic of a mechanical holding mechanism and the mathematical parameters to hold the microneedle device within the socket and allow the microneedle device to move in 3D directions.

### DETAILED DESCRIPTION

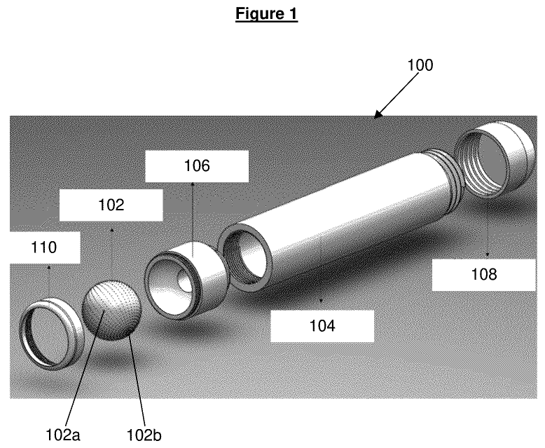

In general, the present disclosure relates to a microneedle apparatus 100 as shown in FIG. 1 for engaging a surface, and uses thereof.

The microneedle apparatus 100 according to various embodiments of the present disclosure offers many advantages. For example, the microneedle apparatus 100 may be used on any area or surface effectively and may be rolled on skin in any direction. This is in contrast to conventional microneedle apparatus, which are limited to be used on certain skin surfaces and areas only and allow one dimensional movement (pressing down only, as in the case of a microneedle patch or microneedle stamp) or two-dimensional movement (pressing down and rolling in one direction, as in the case of a derma roller). The microneedle device 102 may be pressed down and rolled in any directions on the surface, thereby achieving three-dimensional movement. The microneedle apparatus 100 may be shaped like a pen, or handle, or stylus, or any hand-held device.

According to various embodiments of the present disclosure as shown in FIG. 1, there is provided a microneedle apparatus 100 for engaging a surface.

In a particular embodiment, in use, the plurality of microneedles 102b form openings on the surface.

In a particular embodiment, a substance, a mixture, or a formulation may be subsequently delivered to the surface via the openings. In a particular embodiment, in use, the microneedle device 102 may deliver a substance to the surface via the openings. In this way, the microneedle apparatus 100 may be for delivering a substance, such as a therapeutic and/or cosmetic agent, to a surface, such as skin. For the purposes of the present disclosure, a substance may be taken to comprise a compound, a mixture, or a formulation thereof.

In a particular embodiment, the apparatus 100 may include suitable sensors, which may be configured to detect one or more properties of the surface, for example, surface electrical properties including but not limited to electrical resistivity, electrical conductivity; surface chemical properties including but not limited to pH, acidity; surface physical properties including but not limited to pressure, surface roughness, hardness, adhesion, friction, Young's modulus, or a combination thereof. The apparatus 100 may be used to detect bulk properties on the surface and/or underneath the surface, and/or properties of a substance on the surface and/or underneath the surface, for example, density, concentration of a substance, chemical or thermodynamic activity of a substance, bulk electrical resistivity or conductivity, bulk pH, bulk pressure, capacitance, or the like. The apparatus 100 may be used to detect body fluid properties or blood properties including but not limited to biological markers, enzymes, glucose, chemical activity of a substance therein, pH, electrolytes, blood gas, and the like.

The apparatus 100 comprises a housing 104 comprising a socket 106, and a microneedle device 102 moveable within the socket 106. The microneedle device 102 comprises a substantially spherical substrate 102a and a plurality of microneedles 102b projecting from the substrate. The microneedle device 102 is configured to move within the socket 106 and against the surface with 3-dimensional movements such that, in use, the plurality of microneedles 102b engages the surface, or form openings on the surface, and may thereby deliver the substance, the mixture, or the formulation to the surface via the openings.

The housing 104 may be of any suitable shape and size for a user to hold while the microneedle apparatus 100 is in use. For example, the housing 104 may be elongated, tapered, curved, symmetrical, asymmetrical, or any combination thereof. The housing 104 may have a cylindrical shape, or a combination of cylindrical shapes with varied diameter connecting at one end to each other via smooth joints. In a particular aspect, the housing 104 may be cylindrical, or may comprise a plurality of cylindrical-shaped bodies connected at their respective ends. For example, the housing 104 may be 0.1-50 cm in height. The apparatus 100 may comprise a housing cap 108 to close the housing 104.

The microneedle apparatus 100 contains a socket 106 to hold the microneedle device 102. The socket 106 may provide holding mechanism to support 1-dimensional, 2-dimensional or 3-dimensional movements of the microneedle device 102. The socket 106 may be any suitable hollow/concave member or a cavity into which a microneedle device fits. The apparatus 100 may further comprise a socket ring 110 to secure the microneedle device 102 to the socket 106. For example, the socket 106 may be a ball socket The socket 106 may have an external body with one or more internal openings. The one or more internal openings may be substantially spherical in a concave manner to accommodate the substantially spherical substrate 102a. The one or more internal openings may have an open angle of up to 180°. The socket 106 may be made of any suitable materials with or without functionalisation, including but not limited to metals, metal alloys, metal oxides, polymers, cellulose, ceramic, glass, plastics, rubbers, woods, 20 carbon-based materials, silicone-based materials, polymer-based materials, inorganic or organic materials, composites, conjugates, or a combination thereof.

The microneedle device 102 comprises the substantially spherical substrate 102a and microneedles 102b projecting from the substrate 102a. In a particular embodiment, the microneedle device 102 is used to engage the surface to form openings on the surface such as by penetrating the surface, which can be skin or tissue or mucosa or other biological membranes or other surfaces. The microneedle apparatus 100 may be used for local delivery, mucosal delivery, transdermal delivery of substances such as a therapeutic and/or cosmetic agent. In use, the microneedle device 102 is moved within the socket 106 and against the surface with 3-dimensional movements, and thus may be used for creating openings, such as micro-pores, micro abrasions within and/or on the surface before, during, or after the substances are applied. The openings facilitate delivery of the substances to the surface.

For the purposes of the present disclosure, a therapeutic or cosmetic agent may be any substance which exerts a therapeutic or prophylactic or cosmetic effect in a subject to be administered. Any suitable therapeutic and/or cosmetic agent may be used for the purposes of the present disclosure. For example, the therapeutic agent may be, but not limited to an anaesthetic, analgesic, or pain relieving agent such as lidocaine, tetracaine, benzocaine, diclofenac, ketamine, midazolam, propofol, an anti-inflammatory agent, an antioxidant, amino acids, amines, amides, acids, protective or regenerative agents, an anti-proliferative agent, an anti-cancer agent, a growth factor, a biologic drug, RNA, DNA, peptides, protein, lipoprotein, or a combination thereof, and the cosmetic agent may be, but not limited to, hyaluronic acids, pullulans, collagen, peptides, vitamins, exosomes, secretomes, derived vesicles of natural or synthetic origin, skin care active compounds, moisturizing agents, regenerating, rejuvenating or antiaging agents, oils, ceramides, lipids, lipid derivatives, or a combination thereof. In particular, the therapeutic agent may be a topical, epidermal, dermal, mucosal, or transdermal delivered drug, extract, molecule, or mixture. For the purposes of the present disclosure, a formulation may be any combination of various ingredients or a mixture of chemical substances for any particular purpose.

For the purposes of the present disclosure, 1-, 2-, or 3-dimensional movement is defined as movement on a line, a plane, or a space, respectively. An example of 1-dimensional movement is the movement on a straight line, an example of 2-dimensional movement is the movement on a plane, and an example of 3-dimensional movement is the movement in a space without any constraint. Movement in a space with constraint of a fixed straight line is 1 dimensional movement, movement in a space with constraint of a fixed plane is 2 dimensional movement. Movement in a space without any constraint is 3 dimensional movement, or where movement happens multi-directionally.

As an example, 3-dimensional movements of the microneedle device 102 against the surface may comprise surge (forward and/or backward movement on the X-axis parallel to the surface), sway (left and/or right movement on the Y-axis parallel to the surface and perpendicular to the X-axis), heave (upward and/or downward movement on the Z-axis perpendicular to the surface or XY-plane), roll (side to side tilting on the X-axis), pitch (forward and/or backward tilting on the Y-axis), yaw (left and/or right turning on the Z-axis), and/or a combination thereof. The microneedle device 102 may have high skin penetration and be moved easily in 1 dimension, 2 dimensions, or 3 dimensions. In particular, the microneedle device 102 can be rolled easily in 3 dimensions along the surface. Accordingly, the microneedle apparatus 100 may be used easily on skin without the limitation of the varied skin surface, skin condition or even the direction of movement of the microneedle device 102. In this way, the microneedle apparatus 100 advantageously allows the user to easily perform microneedling on various parts of their skin, even on hard-to-reach areas.

The microneedle device 102 may be integral with the socket 106 and cannot be removed. Alternatively, the microneedle device 102 may be a removable from or removably attachable to the socket 106. The microneedle device 102 may be detachable from the socket 106 for ease of change or replacement. The microneedle device 102 may be dissolvable. In this way, a user can conveniently replace the microneedle device 102 before or after each use of the apparatus 100. The socket 106 may be removable, replaceable and/or reusable. Accordingly, the microneedle device 102 may be suitable for single-use only. In this way, there is no issue of improper cleaning after use and therefore would eliminate any risk of contamination and infection. The sterility of the microneedle device 102 may also be maintained. The microneedle device 102 may also be kept sterile using individual sterile packaging. Any risk of infection can be prevented as it may be disposed after use. The microneedle device 102 may be rolled inside the socket 106 upon application of the microneedle device 102 on the surface such as skin or mucosa, or biological membranes.

In some embodiments, the microneedle apparatus 100 further comprises a reservoir connectable to or integrated with the housing 104, wherein the reservoir is configured to store the substance, the mixture, or the formulation. The reservoir may be contained within the housing 104, or may be an external reservoir to be connected to the housing 104. In this way, the microneedle apparatus 100 may also be connected with available skin care products/container, which may include but not limited to tubes or containers of varied sizes and shapes. For example, the reservoir may be in the form of a bottle, a vial, a jar, a stick, or a tube. The microneedle apparatus 100 may include an adaptor for connecting it to other tubes/containers. For example, the tubes/containers may be connected to the housing 104 via the adaptor.

The reservoir may be configured to dispense the substance to the microneedle device 102 via the socket 106. The microneedle device 102 may comprise at least one opening in fluid connection with the reservoir to receive the substance from the reservoir. The socket 106 is likewise in fluid communication with the reservoir and the microneedle device 102. Accordingly, the socket 106 may comprise at least one opening in fluid connection with the reservoir and/or at least one opening in fluid connection with the microneedle device 102. In this way, the microneedle device 102 is capable of receiving the substance from the reservoir via the socket 106. The fluid connection may be any suitable connection that allows the substance to pass from the reservoir to the microneedle device. For example, the fluid connection may be a direct connection through aligned openings in the reservoir and the microneedle device 102, or via a tube, a pipe, or any suitable structure that allows the substance to pass through.

The microneedle device 102 may be capable of delivering the substance to the surface. In particular, the substrate 102a of the microneedle device 102 may comprise pores configured to deliver the substance through the pores to the surface, and/or the plurality of microneedles 102b may be configured to deliver the substance through the microneedles 102b to the surface. The microneedle apparatus 100 and/or the microneedle device 102 may comprise a vibration element. For example, the vibration element may be a motor, a piezoelectric element, or the like. In this way, the microneedle device 102 may be capable of vibrating when the apparatus 100 is in use, so as to enhance the forming of openings on the surface and increase efficacy of substance delivery. Vibration of the microneedle device 102 may be made with any suitable controlled frequency. The frequency may be 1-200000 times per minute. In particular, the frequency may be 100-190000 times per minute, 1000-180000 times per minute, 2000-170000 times per minute, 3000-160000 times per minute, 4000-150000 times per minute, 5000-140000 times per minute, 6000-130000 times per minute, 7000-120000 times per minute, 8000-110000 times per minute, 9000-100000 times per minute, 10000-90000 times per minute, 15000-85000 times per minute, 20000-80000 times per minute, 25000-75000 times per minute, 30000-70000 times per minute, 35000-65000 times per minute, 40000-60000 times per minute, 45000-55000 times per minute. Even more in particular, the frequency may be 6,000-60,000 times per minute.

In some embodiments, instead of or in addition to receiving the substance from the reservoir, the microneedle device 102 may comprise the substance. For example, the substrate 102a comprises the substance. The substance may include a therapeutic and/or cosmetic agent. The therapeutic agent may be an anaesthetic or pain relieving agent such as, but not limited to, lidocaine, tetracaine, benzocaine, diclofenac, ketamine, midazolam, propofol, an anti-inflammatory agent, an anti-proliferative agent, an anti-cancer agent, a growth factor, a biologic drug, RNA, DNA, peptides, protein, or a combination thereof. The cosmetic agent may be, but is not limited to, hyaluronic acid, collagen, peptides, vitamins, skin care active compounds, moisturizing agents, rejuvenating or antiaging agents, oils, lipids, lipid derivatives, or a combination thereof. In particular, the therapeutic agent may be a topical or transdermal delivered active agent.

The substance comprised in the microneedle device 102 may be of any suitable form. For example, the substance may be coated on, encapsulated into, injected into, or placed into the microneedle device 102. The coating may be formed by any suitable method. For example, the coating comprising the substance may be applied on the microneedle device 102 by, but not limited to, spray coating, dip coating, dry coating, electrostatic coating, and the like or the combination thereof. For example, the coating may be formed on each of the plurality of microneedles 102b of the microneedle device 102, and/or only on some of the microneedles 102b, and/or on the substrate 102a of the microneedle device 102. The coating may be formed only on a tip part of the microneedle 102b or the entire surface of the microneedle 102b.

The microneedle device 102 may be held in the socket 106 via any suitable holding mechanisms. In particular, the substrate 102a is held in the socket 106 by chemical, physical, mechanical, magnetic, electrical mechanisms, or a combination thereof, to hold the microneedle device 102 within the socket 106 and support the movement of the microneedle device 102.

In one embodiment, the microneedle device 102 may be held in the socket 106 mechanically, wherein n≥1000Rρ/σA, wherein n is the density of the microneedles 102b, R is the inner radius of the socket 106, p is the mass density of the microneedle device 102, o is the compressive strength of the microneedle device 102, and A is the contact area of the tips of the microneedles 102b with the socket 106. The mechanical mechanisms may include a cavity or a concave compartment or any mechanical supports to secure the microneedle device 102 therein, thereby allowing the microneedle device 102 to be capable of moving in 1D, 2D and 3D therein.

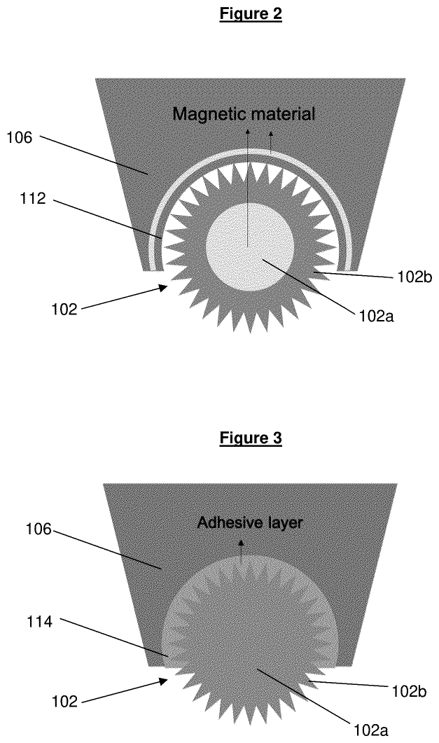

In one embodiment as shown in FIG. 2, the microneedle device 102 and socket 106 may comprise magnetic materials or layers 112, or may be made wholly or partly using magnetic materials, and the magnetic forces will secure the microneedle device 102 to the socket 106. For example, the core of the substrate 102a of the microneedle device 102 is formed of a magnetic material. The magnetic material may be a ferromagnetic material, a ferrimagnetic material, or a paramagnetic material. For example, the magnetic material may be iron, nickel, cobalt, magnetite, rare-earth metals, lodestone, aluminium, platinum, alloys thereof, or combinations thereof.

In one embodiment as shown in FIG. 3, the socket 106 may comprise an adhesive or holding material or layer 114 for holding the microneedle device 102 in the socket 106. The adhesive or holding layer 114 may comprise any suitable material which allows 3-dimensional movement of the microneedle device 102 within the socket 106. The adhesive or holding material 114 may comprise any adhesive material that is suitable for contacting with skin. For example, the adhesive or holding material 114 may comprise acrylic, hydrocolloid, silicone, hydrogel, slime, paste, or a combination thereof.

As described above, the microneedle device 102 comprises a plurality of microneedles 102b that project from the substrate 102a. The microneedles 102b are micro projections or micro-projection-like structures that can refer to anything with any shape at microscale at least in one dimension that extend from the substrate 102a. The substrate 102a or a portion of the substrate 102a may be or comprise a sphere, a substantially spherical shape, a cube, a rectangular prism, a triangular prism, a cylinder, a pyramid, a cone, a torus, a dumbbell, a tetrahedron, pentahedrons, hexahedrons, octahedrons, or any polyhedron, irregular shapes.

Micro-projection-like structures refer to anything that contains micro projections. For example, a cubic crystal can be defined as a micro-projection-like structure with 4 micro projections at 4 corners, a stellated regular or irregular octahedron is a micro-projection-like structure with 8 micro projections at 8 corners, and a stellated regular/irregular polyhedron is a micro-projection-like structure with many micro projections at their corresponding corners.

The substantially spherical substrate 102a is a body which supports the plurality of microneedles 102b projecting from it. For the purposes of the present disclosure, substantially spherical refers to a degree of deviation that is sufficiently small so as to not measurably detract from identifying the substrate 102a as a sphere. In particular, the term substantially spherical may include a substrate which is spherical or nearly-spherical. Spherical or near-spherical may comprise substrates which are suitable for 3D printing and which have dimensions which have a low aspect ratio and avoid jagged or irregular shapes. For example, the substantially spherical substrate 102a may have an aspect ratio which is ≤4. In particular, the aspect ratio of the substrate 102a may be ≤1.2. Aspect ratio may be defined as a ratio of the longest dimension of the substrate and the shortest dimension of the substrate 102a.

The substrate 102a is substantially spherical or has a dumbbell or multi-balls shape, or combinations thereof.

The substrate 102a may be of any suitable size. For example, the average diameter of the substrate 102a may be 0.05-1000 mm. In particular, the average diameter may be 0.1-900 mm, 0.2-800 mm, 0.3-700 mm, 0.4-600 mm, 0.5-500 mm, 1-400 mm, 10-300 mm, 50-250 mm, 100-200 mm. Even more in particular, the diameter of the substrate 102a may be 0.5-50 mm.

The substrate 102a may be comprise any suitable material including but not limited to metals, metal alloys, metal oxides, polymers, cellulose, ceramic, glass, plastics, rubbers, wood, carbon-based materials, silicone-based materials, polymer-based materials, composites, or a combination thereof. The substrate 102a may be solid, hollow, and/or may have an opening for injecting substances therein, and/or may have pores on the substrate exterior to facilitate delivery of the substance to the surface.

The substrate 102a may further comprise suitable additives to improve the functional properties or to improve device functionality. For example, the additive may be a material to improve mechanical strength of the microneedles 102b. In particular, the additive may be hydrogel, salts, calcium phosphate, calcium carbonate, silica, graphene, carbon nanotubes, nanocrystals, nanoparticles, micro particles, or a combination thereof.

The microneedle device 102 comprises multiple suitably sized and shaped microneedles 102b. The microneedles 102b may be hollow or solid microneedles 102b. According to a particular aspect, each of the plurality of microneedles 102b may have the same or different height (length). In particular, the height may be a suitable height so as to achieve administration of the substance such as a therapeutic or cosmetic agent, but at the same time short enough to avoid contacting nerves in the skin so as to reduce the possibility of pain and avoid the possibility of bleeding. The height of the microneedle 102b also depends on the surface on which the microneedle device 102 is to be used. For example, the average height of each of the plurality of microneedles 102b may be 0.1-10000 μm. In particular, the height of each of the plurality of microneedles 102b may be 0.1-1 μm, 0.1-300 μm, 1-4900 μm, 100-4800 μm, 200-4700 μm, 300-4600 μm, or 400-4500 μm, 500-4400 μm, 600-4300 μm, 700-4200 μm, 800-4100 μm, 900-4000 μm, 1000-3900 μm, 1100-3800 μm, 1200-3700 μm, 1300-3600 μm, 1400-3500 μm, 1500-3400 μm, 1600-3300 μm, 1700-3200 μm, 1800-3100 μm, 1900-3000 μm, 2000-2900 μm, 2100-2800 μm, 2200-2700 μm, 2300-2600 μm, or 2400-2500 μm. Even more in particular, the height may be 10-500 μm.

Each of the plurality of microneedles 102b may have the same or different shape. The plurality of microneedles 102b projecting from the substrate 102a may be a needle shape or a structure including a needle shape. However, for the purposes of the present disclosure, the microneedle 102b may not be limited to a structure of a needle shape having a tapered tip, and may comprise a structure lacking a tapered tip. Each of the plurality of microneedles 102b may be conical or pyramidal, so long as they have the capability to pierce a surface such as skin, or tissue, or mucosa. For example, each of the plurality of microneedles 102b may have a conical shape, or a polygonal pyramid shape such as a triangular pyramid, square pyramid or in other suitable shapes.

The microneedles 102b of the microneedle device 102 may be disposed spaced apart. For example, the microneedle device 102 may have a suitable microneedle density. A suitable microneedle density is required to achieve efficient piercing of the skin and to ensure that the microneedles 102b do not break when moving within the socket 106. However, if the microneedle density is too high, maintaining the strength of the microneedles 102b may become difficult. The microneedles 102b of the microneedle device 102 may be hollow, and/or may have an opening within the microneedles 102b to facilitate delivery of a substance to the surface.

The microneedle device 102 may be made of any suitable material. The microneedles 102b or micro-projections may be made of same or different materials from the substrate 102a, which may be detachable or fixed onto the substrate 102a. For example, the microneedle device 102 may comprise plastic, hydrogels or organogels, gelatin, gelatin methacrylate hydrogel, hyaluronic acid, silicone, polymer, sugar, glass, ceramic metal, metal alloys, metal oxides, and/or composites.

The polymer may be any suitable polymer. For example, the polymer may be, but not limited to, cellulose, cellulose derivatives, cellulose hybrids, cellulose composites, Eudragit, polymethacrylate-based copolymers, resomers, polyesters, pullulans, silica based polymers, zeins, kerateins, acrylates, polycarbonates, polycaprolactones, carboxymethylcellulose (CMC) or sodium CMC, amylopectic, poly (methylmetha-acrylate) (PMMA), poly-L-lactic acid (PLA), polyglycolic acid (PGA), polylactic-co-glycolic acid (PLGA), cyclic-olefin polymer, poly (vinyl pyrrolidone) (PVP), polyethylene glycol diacrylate (PEGDA), or polyethylene glycol (PEG)-fibrinogen hydrogel.

The sugar may be, but not limited to, galactose, maltose, or dextrin.

The metal, metal alloys, or metal oxides may be, but not limited to, copper, brass, iron, iron oxide, magnesium, manganese, silver, gold, platinum, aluminium, aluminium oxides, cobalt, chromium, chrome, cobalt chrome, stainless steel, titanium, titania, tantalum, palladium, palladium-cobalt alloys, nickel, nitinol, zirconium, or zirconia.

According to a particular aspect, the gelatin comprised in the gelatin and/or gelatin methacrylate hydrogel may be derived from: porcine, bovine, fish sources, or a combination thereof. In particular, the gelatin may be derived from porcine or fish sources. Even more in particular, the gelatin was derived from porcine or fish skin.

The microneedle device 102 may comprise suitable additives. In particular, the gelatin, gelatin methacrylate hydrogel, hyaluronic acid and/or polymer may comprise additives. Even more in particular, the gelatin methacrylate hydrogel may comprise suitable additives. For example, the additive may be a mechanical strengthening additive. In particular, the additive may be calcium phosphate, calcium carbonate, silica, graphene, carbon nanotubes, quantum dots, nanocrystals, or a combination thereof.

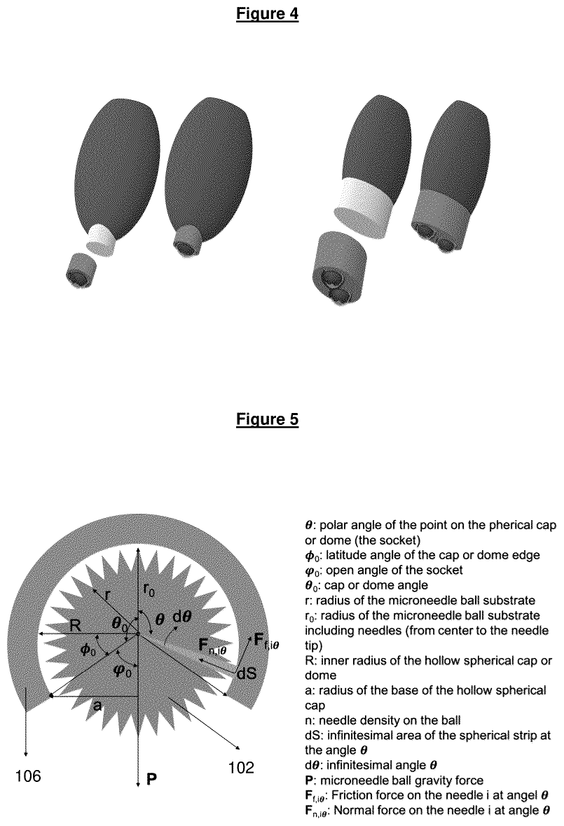

In some embodiments as shown in FIG. 4, the microneedle apparatus 100 may comprise one or more sockets 106 to each contain a microneedle device 102. The apparatus 100 may include one or more reservoirs to contain different substances or formulations for various applications. The reservoirs may be detachable from the apparatus 100 for ease of change or replacement or for cleaning purpose, or may be permanently attached to the apparatus 100 or part of the apparatus 100. One or more reservoirs of multiple shapes or multiple patterns may be connected to the apparatus 100 for combination treatment. One or more substances or formulations may be loaded into the reservoirs for combination treatment. The apparatus 100 may further comprise one or more adapters to allow the apparatus 100 to be attached to external reservoirs, for example, cartridges, bottles or any containers. The one or more sockets 106 may comprise any suitable material. For example, the one or more sockets 106 may comprise metals, metal alloys, metal oxides, polymers, cellulose, ceramic, glass, plastics, rubbers, woods, carbon-based materials, silicone-based materials, polymer-based materials, composites, hybrid materials, nanohybrids, or a combination thereof.

The microneedle apparatus 100 may comprise a plurality of sockets 106 and a plurality of microneedle devices 102, wherein a microneedle device 102 is held in each of the plurality of sockets 106, each microneedle device 102 comprising a substantially spherical substrate 102a and a plurality of microneedles 102b projecting from the substrate 102a, wherein each microneedle device 102 is configured to move within each of the plurality of sockets 106 with 3-dimensional movements. In one embodiment, the microneedle device 102 may comprise at least two sockets 106 and at least two microneedle devices 102, each microneedle device 102 being held in each socket 106. Each socket 106 may have one or more openings to allow the substance to flow through. Each microneedle device 102 may have one or more openings to allow a substance to flow through and subsequently to the surface.

The microneedle apparatus 100 may comprise one or more microneedle devices 102 and a holder or handle with holding compartment or holding space or holding mechanism to hold the microneedle devices 102 in place without restricting its movement, or restricting the movement partially or completely in certain space.

The microneedle apparatus 100 of the present disclosure may be used as or in a delivery system, a sensing system, or a collecting system. In particular, the apparatus 100 may be used to enhance the absorption of active ingredients through varied administration routes including but not limited to transdermal, transepidermal, epidermal, oral, sublingual, nasal, respiratory, ocular, retinal, subretinal, intravenous, intraoperative administration.

The microneedle apparatus 100 may comprise one or more compartments acting as reservoirs to contain various substances or formulations, for example, active or base ingredients or formulations of various ingredients or substances. These compartments may be connected to a mechanism to dispense the ingredients during application of the microneedle apparatus 100. Dispensing mechanisms may be triggered by the microneedle device rolling upon the surface during application, or by a trigger or button/buttons, touch sensors, force sensors, etc.

The microneedle devices 102 may be used for penetrating skin, tissue, mucosa or gums. In particular, the microneedle devices 102 may be suitable for topical or transdermal delivery of therapeutics or cosmetic agents.

According to a particular aspect, the kinetic friction coefficient between tip of the microneedles 102b and the inner surface of the socket 106 may be ≤2. The static friction coefficient between tip of the microneedles 102b and the inner surface of the socket 106 may be ≤2.

According to some embodiments of the present disclosure, there is a kit comprising the microneedle apparatus 100 as described above, and the substance. The substance may include one or more therapeutic and/or cosmetic agents. The therapeutic agent may be, but not limited to, an anaesthetic or pain relieving agent such as lidocaine, tetracaine, benzocaine, diclofenac, ketamine, midazolam, propofol, an anti-inflammatory agent, an anti-proliferative agent, an anti-cancer agent, growth factors, biologics, RNA, DNA, peptides, proteins, or a combination thereof, and the cosmetic agent may be, but not limited to, hyaluronic acid, collagen, peptides, vitamins, skin care active compounds, moisturizing agents, rejuvenating or antiaging agents, oils, ceramides, lipids, lipid derivatives, or a combination thereof. In particular, the therapeutic agent may be a topical or transdermal delivered active agent. In this way, a user can conveniently use the apparatus with the one or more therapeutic or cosmetic agent provided in the kit. The kit may be customised to a specific user.

A method of using the microneedle apparatus 100 to engage a surface comprises:
handling the microneedle apparatus 100 comprising a housing 104 with a socket 106;
moving a microneedle device 102 within the socket 106 and against the surface with 3-dimensional movements, the microneedle device 102 comprising a substantially spherical substrate 102a and a plurality of microneedles 102b projecting from the substrate 102a; and
engaging the surface via the plurality of microneedles 102b.

The method may further comprise forming openings on the surface with the plurality of microneedles 102a as the microneedle device 102 moves against the surface, and delivering a substance to the surface via the openings formed on the surface. In a particular embodiment, the delivering may be via the microneedle device 102.

The method may further comprise detecting one or more surface properties comprising electrical resistance, electrical conductivity, surface pressure, or a combination thereof. The method may further comprise detecting one or more substance properties comprising density, pH, pressure, concentration, chemical or thermodynamic activity, electrical resistivity or conductivity, capacitance, or a combination thereof.

The moving of the microneedle device 102 within the socket 106 and against the surface with 3-dimensional movements may be for any suitable period of time. For example, the moving may be for 4 hours. In particular, the moving may be for 1 hour, 1.5 hours, 2 hours, 2.5 hours, 3 hours, 3.5 hours, or 4 hours. Even more in particular, the moving may be for 1 hour. In this way, the forming of openings on the surface and delivering of the substance to the surface are achieved in an efficient manner whilst maximising delivery of the substance and/or until all the substance has been completely delivered to the surface.

A method of making the microneedle apparatus 100 may comprise 3D printing various parts of the apparatus 100, particularly the microneedle device 102, using any suitable material. The method may comprise micromoulding or moulding of various parts of the apparatus 100. The moulding or micromoulding method can utilize 3D printing method wherein positive moulds of various parts of the apparatus can be 3D printed directly, followed by a negative mould preparation from the positive moulds. A negative mould can be prepared by casting mouldable materials onto the 3D printed moulds (the positive moulds), followed by curing the mouldable materials to form a negative structure of the printed moulds, then removing the 3D printed mould from the cured negative mould. The method may comprise microfabrication techniques, lithography, soft lithography, film deposition, etching, bonding, and micromoulding, injection moulding, compression moulding, compressing, compress cutting and combination thereof. In an example of indirectly 3D printing the microneedle device 102, the method may comprise one or more of the following:
(a)(i) providing a 3D printed mould of the microneedle device 102, wherein the mould of microneedle device 102 contains a substrate 102a and a plurality of microneedles 102b projecting from the substrate 102a, wherein the substrate 102a of the mould comprises a sphere, a substantially spherical shape, a cube, a rectangular prism, a triangular prism, a cylinder, a pyramid, a cone, a torus, a dumbbell, a tetrahedron, pentahedrons, hexahedrons, octahedrons, or any polyhedron, irregular shapes;
(ii) casting a 3D printed mould with a precursor solution or mouldable material;
(iii) curing the precursor solution or mouldable materials to form a negative mould of the microneedle device 102;
(iv) removing the 3D printed mould from the cured negative mould;
(v) casting the negative mould with suitable materials to form the microneedle device 102;
(b) or compressing materials into a mould, or compression cutting or compression moulding of materials in any form which may include but not limited to solid, liquids, semi solids, sheets, elastic or plastic materials (such as rubber), powders or combination thereof, wherein the compression may be direct or indirect using hard moulds or flexible moulds or using punches, wherein compression cutting or compression punching or compress moulding may involve compression and/or cutting or moulding of suitable materials described in present disclosure.

The precursor solution or precursor material may be a hydrogel precursor solution, or a monomer, a polymer precursor solution, a curable solution, a metal precursor, a metal alloy precursor, a metal oxide precursor, a cellulose precursor, a ceramic precursor, a glass precursor, a plastics precursor, a rubbers precursor, a wood precursor, a carbon-based precursor, a silicone-based precursor, a polymer-based precursor, a composite precursor, or a combination thereof. In particular, the precursor solution may comprise a photo-curable solution and a photoinitiator. Any suitable photoinitiator may be used for the purposes of the present disclosure.

The precursor solution may further comprise a suitable additive or additives. For example, the additive may be a mechanical strengthening material or functional material to improve mechanical strength or functional properties. In particular, the additive may be calcium phosphate, calcium carbonate, silica, graphene, carbon nanotubes, quantum dots, nanoparticles, micro particles, nanocrystals, or a combination thereof. The functional material may be any suitable material to alter surface properties of the microneedle device 102 including but not limited to, hydrophilicity, hydrophobicity, static surface charge, acidity, or for controlled release of a therapeutic agent or cosmetic agent from the microneedle device 102.

The microneedle apparatus 100 according to embodiments of the present disclosure offers several advantages over previous microneedle devices or apparatuses. For example, the microneedle apparatus 100 may be used on any area or surface with better effectiveness and may be moved and/or rolled on skin surfaces in multiple directions that may not be restricted. This is in contrast to conventional microneedles or microneedle devices, which has limited use on certain skin surfaces and areas only, and allow only one dimensional movement (pressing only, for a microneedle patch or stamp) or two-dimensional movement (pressing and rolling, for a derma rollers). The substrate 102a of the microneedle device 102 of the apparatus 100 may be pressed down and rolled in all directions that may not be restricted, thereby achieving three-dimensional or multi-dimensional movement.

Area of the infinitesimal strip dS of the microneedle device 102 at the infinitesimal angle θ:

dS=2πR² sin θdθ

Number of microneedles on the strip dS:

N_{θ}=n2πR² sin θdθ

Forces exerted on the microneedle device 102:

$${\sum }_{θ} {\sum }_{i = 1}^{{N}_{θ}} {F}_{n , i θ} + {\sum }_{θ} {\sum }_{i = 1}^{{N}_{θ}} {F}_{f , i θ} + P = 0 \qquad ( 1 )$$

Assuming that all the microneedles 102b are of equal length, identical structure and properties, due to the symmetry of the spherical substrate 102a, the normal force on all microneedles 102b lying within the strip dS has the same magnitude F_{n,i} with different directions, and the friction force Fr, i also has the same magnitude. Thus:

$${\int }_{\frac{π}{2}}^{\frac{π}{2} + {∅}_{0}} {N}_{θ} {F}_{n , i θ} + {\int }_{\frac{π}{2}}^{\frac{π}{2} + {∅}_{0}} {N}_{θ} {F}_{f , i θ} + P = 0 \qquad ( 2 )$$

Project equation (2) to the direction of P:

$${\int }_{\frac{π}{2}}^{\frac{π}{2} + {∅}_{0}} {F}_{n , i θ} ( - cos θ ) n 2 π {R}^{2} sin θ d θ + {\int }_{\frac{π}{2}}^{\frac{π}{2} + {∅}_{0}} {kF}_{n , i θ} sin θ n 2 π {R}^{2} sin θ d θ - P = 0 \qquad ( 3 )$$

Where k is the coefficient of friction,

$${\int }_{\frac{π}{2}}^{\frac{π}{2} + {∅}_{0}} sin θcos θ d θ = {\int }_{\frac{π}{2}}^{\frac{π}{2} + {∅}_{0}} sin θ d ( sin θ ) = - \frac{{sin}^{2} {∅}_{0}}{2} \qquad ( 4 )$$

$${\int }_{\frac{π}{2}}^{\frac{π}{2} + {∅}_{0}} {sin}^{2} θ d θ = \frac{{ϕ}_{0}}{2} + \frac{sin 2 {ϕ}_{0}}{4} \qquad ( 5 )$$

Then it follows that:

$${F}_{n} = \frac{P}{n 2 π {R}^{2} [ \frac{{sin}^{2} {ϕ}_{0}}{2} + k ( \frac{{ϕ}_{0}}{2} + \frac{sin 2 {ϕ}_{0}}{4} ) ]} = \frac{( \frac{4}{3} π {R}^{3} ρ + 4 π {R}^{2} {nV}_{n eedle} ρ ) g}{n 2 π {R}^{2} [ \frac{{sin}^{2} {ϕ}_{0}}{2} + k ( \frac{{ϕ}_{0}}{2} + \frac{sin 2 {ϕ}_{0}}{4} ) ]} {F}_{n} = \frac{2 π ( \frac{R}{3} + {nV}_{n eedle} ) ρ g}{n [ \frac{{sin}^{2} {ϕ}_{0}}{2} + k ( \frac{{ϕ}_{0}}{2} + \frac{sin 2 {ϕ}_{0}}{4} ) ]} \qquad ( 6 )$$

Conditions for the microneedles 102b to tolerate forces without deformation:

$${F}_{n} = \frac{( a R + b n ) ρ g}{n [ \frac{{sin}^{2} {ϕ}_{0}}{2} + k ( \frac{{ϕ}_{0}}{2} + \frac{sin 2 {ϕ}_{0}}{4} ) ]} \leq {F}_{compressive stength} \qquad ( 7 )$$

$${F}_{n} \approx \frac{\frac{4}{3} π {R}^{3} ρ g}{n 2 π {R}^{2} [ \frac{{sin}^{2} {ϕ}_{0}}{2} + k ( \frac{{ϕ}_{0}}{2} + \frac{sin 2 {ϕ}_{0}}{4} ) ]} \leq {F}_{compressive stength} And {F}_{f} = k {F}_{n} \leq {F}_{shear strength} \qquad ( 8 )$$

### Example

The strength of the microneedles 102b can be described as follows with an exemplary example. A microneedle device 102 was formed based on the schematic diagram as shown in FIG. 1. 304 stainless steel was used as the material for the substantially spherical substrate 102a and the plurality of microneedles 102b projecting from the substrate 102a, while steel was used as the material for the socket 106.

Compressive strength σ=310 MPa, tensile strength=620 MPa, shear strength=0.75×tensile strength=465 MPa, ρ=8.06M g/m³, R=1.5 cm, and V_{needle} is infinitesimally small compared with the volume of the substrate 102a. F compressive strength=compressive strength×microneedle tip area, F shear strength=shear strength×microneedle tip area.

As mentioned above, the microneedle device 102 was made of stainless steel, while the socket 106 was made of steel. With the assumption that ϕ₀=45°, the static friction coefficient between stainless steel vs steel=0.8, kinetic friction coefficient=0.42 at clean and dry condition, microneedle length was 250 μm, microneedle tip diameter was A=50 μm, microneedle base diameter was 100 μm.

$$n \geq \frac{\frac{4}{3} π {R}^{3} ρ g}{{F}_{compressive stength} 2 π {R}^{2} [ \frac{{sin}^{2} {ϕ}_{0}}{2} + k ( \frac{{ϕ}_{0}}{2} + \frac{sin 2 {ϕ}_{0}}{4} ) ]}$$

$$n \geq \frac{\frac{4}{3} 1.5 ( 1 {0}^{- 2} ) 8.06 ( 1 {0}^{3} ) 10}{310 ( 1 {0}^{6} ) 3.14 ( 0.0 {5}^{2} ) {( 10 )}^{- 6} [ 0 . {7}^{2} + 0.8 ( \frac{3.14}{4} + \frac{1}{2} ) ]} = 4.3 ( 1 {0}^{- 4} ) ( \frac{microneeclles}{{mm}^{2}} )$$

Based on this result, if 304 stainless steel is the material for the microneedle device 102, the microneedle density of 1 per mm² is sufficient to make sure the microneedle 102b will not be broken sitting inside the socket 106.

From equation (8), the normal force on a single microneedle 102b will increase if the substrate 102a size increases (R increases) or mass density of the substrate 102a increases (ρ increases), or if the open angle of the socket 106 increases (φ₀ increases), or if the microneedle density decreases. To ensure that the microneedles 102b will not be broken inside the socket 106 when the microneedle device 102 is held vertical, an estimation for microneedle density was used: socket with open angle φ₀=80°, or latitude angle ϕ₀=10°, kinetic coefficient of friction k=0.1, R=1 cm, substrate 102a with mass density ρ, microneedle density n, microneedle tip contact area A with the socket 106, and compressive strength σ.

From equation (8), the microneedle density n can be derived as follows:

$$n \geq \frac{1000 R ρ}{σ A} \qquad ( 9 )$$

Materials with mass density, compressive strength and microneedle density satisfying equation (9) should be selected to ensure that microneedles 102b will not be broken inside the socket 106.

With these parameters, the microneedles 102b made of 304 stainless steel were advantageously strong and were not broken when moved inside the socket 106.

The microneedle device 102 may have any suitable microneedle density to achieve efficient piercing of the surface, for example, skin surface or mucosa. The density of the plurality of microneedles 102b on the substrate 102a may be ≥1/dm². In particular, the microneedles density may be ≥1/cm². Even more in particular, the microneedles density may be ≥1/mm².

Whilst the foregoing description has described exemplary embodiments, it will be understood by those skilled in the technology concerned that many variations may be made without departing from the scope of the present disclosure. The scope of the present disclosure as well as the scope of the following claims is not limited to embodiments described herein.

1. A microneedle apparatus for engaging a surface, the apparatus comprising:
a housing comprising a socket; and
a microneedle device moveable within the socket, the microneedle device comprising a substantially spherical substrate and a plurality of microneedles projecting from the substrate,
wherein the microneedle device is configured to move within the socket and against the surface with 3-dimensional movements such that, in use, the plurality of microneedles engage the surface.

2-3. (canceled)

4. The microneedle apparatus according to claim 1, the microneedle device further comprising one or more sensors configured to detect one or more properties of the surface, and/or one or more properties underneath the surface, and/or a substance on the surface and/or a substance underneath the surface.

5. (canceled)

6. The apparatus according to claim 1, wherein the microneedle device is removably attachable to the socket.

7. The apparatus according to claim 3, further comprising a reservoir connectable to or integrated with the housing, wherein the reservoir is configured to store a substance, a mixture, or a formulation.

8. The apparatus according to claim 7, wherein the socket comprises at least one opening in fluid connection with the reservoir, wherein the microneedle device is configured to receive the substance, the mixture, or the formulation from the reservoir via the socket.

9. The apparatus according to claim 1, wherein the substrate of the microneedle device comprises pores configured to deliver a substance, a mixture, or a formulation to the surface.

10. The apparatus according to claim 1, wherein the microneedle device and/or the socket comprises a magnetic material.

11. The apparatus according to claim 1, wherein the apparatus further comprises an adhesive or holding layer between the microneedle device and the socket.

12. The apparatus according to claim 1, wherein the apparatus comprises a plurality of the sockets and a plurality of the microneedle devices, wherein each microneedle device is held in a respective one of the plurality of sockets.

13. (canceled)

14. The apparatus according to claim 1, wherein the substrate has an aspect ratio of ≤1.2.

15. The apparatus according to claim 1, wherein each of the plurality of microneedles has a height of 0.1-10000 μm.

16. (canceled)

17. The apparatus according to claim 1, wherein density of the plurality of microneedles on the substrate is ≥1/dm².

18. The apparatus according to claim 1, wherein the housing is cylindrical, or comprises a plurality of cylindrical-shaped bodies connected at their respective ends.

19. The apparatus according to claim 1, further comprising a vibration element for vibrating the microneedle device.

20. A kit comprising the apparatus according to claim 1, and a substance, a mixture, or a formulation.

21. (canceled)

22. A method of engaging a surface and delivering a substance, a mixture or a formulation to the surface, the method comprising:
handling a microneedle apparatus comprising a housing with a reservoir, a socket, and a microneedle device within the socket, wherein the socket is in fluid connection with the reservoir and wherein the reservoir contains the substance, the mixture or the formulation, further wherein the microneedle device comprises a substantially spherical substrate and a plurality of microneedles projecting from the substrate;
engaging the surface via the plurality of microneedles;
dispensing the substance, the mixture or the formulation from the reservoir to the microneedle device via the socket;
moving a microneedle device within the socket and against the surface with 3-dimensional movements; and
delivering the substance, the mixture or the formulation from the reservoir to the surface via the engagement of the microneedle device onto the surface.

23. The method of claim 22, further comprising:
forming openings on the surface with the plurality of microneedles as the microneedle device moves against the surface; and
delivering the substance, the mixture, or the formulation to the surface via the openings formed on the surface.

24. (canceled)

25. The method according to claim 22, further comprising:
detecting one or more properties of the surface, and/or one or more properties underneath the surface, and/or a substance on the surface and/or a substance underneath the surface.

26. (canceled)

27. The apparatus according to claim 1, wherein the socket has a spherical cavity, and wherein the socket has an open angle of ≤180°.

28. The apparatus according to claim 1, wherein the needle density is configured to satisfy the equation:
$$n \geq \frac{1000 R ρ}{σ A}$$
wherein R is the inner radius of the socket, ρ is mass density of microneedle device, σ is compressive strength of microneedle device, and A is the contact area of the tips of the microneedles with the socket.
